(19) United States
(12) Patent Application Publication
(10) Pub. No.: US 2019/0321567 A1
(43) Pub. Date: Oct. 24, 2019

(54) SINGLE-USE SYRINGE

(71) Applicant: Dmitrii Leonidovich KOROL, Ufa (RU)

(72) Inventor: Dmitrii Leonidovich KOROL, Ufa (RU)

(21) Appl. No.: 16/386,620

(22) Filed: Apr. 17, 2019

(30) Foreign Application Priority Data
Apr. 24, 2018 (RU) 2018115005

(51) Int. Cl. A61M 5/50

(52) U.S. Cl. CPC A61M 5/502; A61M 5/508

(57) ABSTRACT

The invention relates to medical equipment, in particular to single-use devices for injection. The syringe consists of a needle, a barrel, a plunger, and a plunger stopper. The plunger with the plunger stopper has the same length as the syringe barrel and a specific form of a cylindrical handle stop. In order to release the solution, the syringe has not one but several small holes allowing the solution to pass through smoothly, while preventing the plunger from being pushed out from the needle fitting tip. The technical result consists in preventing reuse of the syringe with full immersion of the plunger.

### CROSS-REFERENCE TO RELATED APPLICATIONS

The present application claims priority to Russian patent application RU2018115005 filed on Apr. 4, 2018, which is fully incorporated herein by reference in its entirety.

### FIELD OF THE INVENTION

The invention relates to medical equipment, in particular to single-use devices for injection.

### BACKGROUND

Existing designs described in patents for single-use syringes can be divided into several groups.

Syringes with plunger stoppers fixed at the end of the barrel using a safety snap. Their disadvantages are that the safety snap may fail and this mechanism is complex and expensive to manufacture.

Syringes with the plunger stopper and plunger separated after use. The disadvantage is that separation may occur while drawing the solution.

Syringes with wearing parts. The disadvantage is that the syringe will fail after a specified number of the plunger stopper strokes.

Syringes with a barrel breaking mechanism. The disadvantage of these designs is the lack of a smooth stroke and, in order to inject the solution, one should exert more effort compared to a conventional syringe.

Many designs have a complex or very complex locking mechanism resulting in higher prices and lower reliability.

There is a known single-use syringe (Russian patent No. 2523628, publ. on Jul. 20, 2014) which includes a barrel, a needle, and a piston; it belongs to the first group. This patent describes a non-closed spring mechanism that locks the plunger stopper when the solution is completely injected.

There is a known single-use syringe (Russian patent No. 2358766, publ. on Jun. 20, 2009) which includes a barrel, a needle, the main plunger stopper, a plunger, a plunger stopper, and a material layer. It can be assigned to the second and third group with their inherent drawbacks.

The prototype of the present invention is a single-use syringe which is currently available in pharmacies.

### Objective of the Invention

Prevention of reuse of the syringe following a single complete injection of the solution;

Retaining the usual look and the ease of manufacture of single-use syringes.

### SUMMARY

The invention consists of a needle and a syringe barrel. The barrel, in turn, consists of the following: a plunger stopper, a plunger of a specific shape and length, as well as a protective cap. The syringe can be made with a removable and staked-in needle. The option of the single-use syringe with a removable needle has not one but several small-diameter openings for the solution to be released, which prevents the plunger stopper from being pushed out from the needle fitting tip.

### BRIEF DESCRIPTION OF THE DRAWINGS

The gist of the disclosure is exposed by drawings showing the following:

FIG. 1 shows a side view of a syringe with a staked-in needle.

FIG. 2 shows a side view of a syringe, the option with a removable needle and several holes for the solution to be released.

FIG. 3 shows a side view of a used syringe with a fully plunged plunger stopper.

FIG. 4 shows a side view of the plunger.

FIG. 5 is a perspective view of a plunger stopper with a plunger.

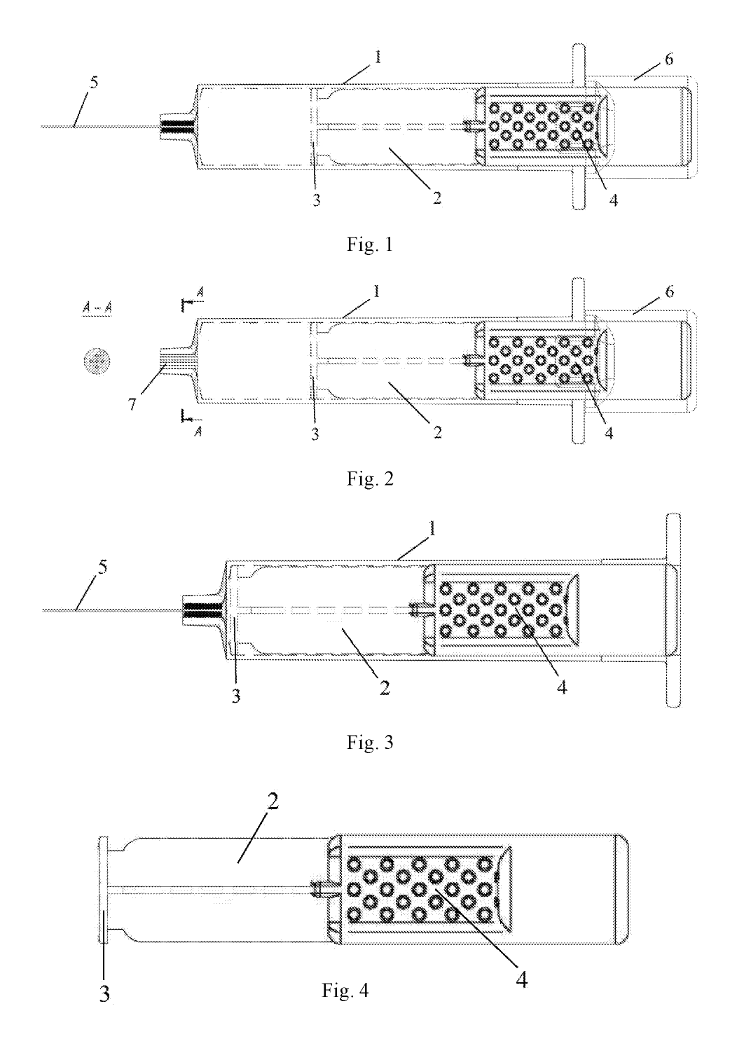

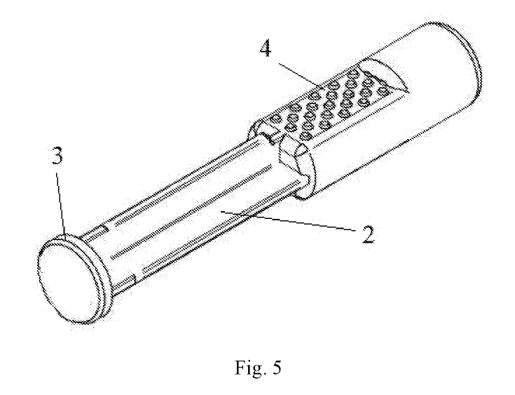

### DETAILED DESCRIPTION OF THE PREFERRED EMBODIMENT

The syringe includes a barrel (1) with a plunger stopper (2) with a plunger (3) in its opening, a needle (5), and a protective cap (6) which prevents the plunger from being pressed on during transportation. On the unload side, the plunger (3) has a cylindrically-shaped handle stop (4) with a diameter equal to the inner diameter of the syringe barrel. To prevent the plunger stopper from being pushed out with the plunger in the version of a syringe with a removable needle, there is not one but several holes (7) used.

The syringe works as follows. Before using the syringe, the protective cap (6) is to be removed. The design of the single-use syringe allows multiple drawings of the solution and a single complete injection of the solution.

Therefore, the present invention allows to prevent reuse of a syringe following a complete injection of the solution, as well as to retain the usual look and the ease of manufacture of available single-use syringes.

The designations adopted in the drawings:

1—barrel,

2—plunger,

3—plunger stopper,

4—plunger's handle stop,

5—needle,

6—protective cap,

7—openings for solution release.

What is claimed is:

1. A single-use syringe, comprising a needle, a barrel, a plunger stopper with a plunger, and a handle stop which is cylinder-shaped with a diameter equal to an inner diameter of the barrel, which does not allow the plunger to be moved to its operational position after complete injection of a solution, wherein the syringe has more than one openings for the solution to be released thus preventing the plunger stopper with a plunger from pushing on a side of a needle fitting.
